(19) United States
(12) Patent Application Publication
(10) Pub. No.: US 2010/0087923 A1
(43) Pub. Date: Apr. 8, 2010

(54) IMPLANTS FOR FACET JOINT REPAIR AND METHODS USE

(76) Inventor: M. Samy Abdou, San Diego, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/546,564

(22) Filed: Aug. 24, 2009

Related U.S. Application Data
(60) Provisional application No. 61/189,750, filed on Aug. 23, 2008.

(51) Int. Cl. A61F 2/44; A61B 17/56

(52) U.S. Cl. 623/17.11; 606/86 R

(57) ABSTRACT

An orthopedic implant is positioned within the facet joint and is attached to the posterior surface of the SAP (superior articular process) of the inferior vertebral bone or onto the anterior surface of the IAP (inferior articular process) of the superior vertebral bone. The implant increases the distance between the IAP and SAP surfaces that make up a facet joint and reduce the joint laxity and at least partially correct the spondylolisthesis produced by a degenerative process.

### REFERENCE TO PRIORITY DOCUMENT

This application claims priority of co-pending U.S. Provisional Patent Application Ser. No. 61/189,750 filed Aug. 23, 2008. Priority of the aforementioned filing date is hereby claimed and the disclosure of the Provisional patent application is hereby incorporated by reference in its entirety.

### BACKGROUND

Progressive constriction of the central canal within the spinal column is a predictable consequence of aging. As the spinal canal narrows, the nerve elements that reside within it become progressively more crowded. Eventually, the canal dimensions become sufficiently small so as to significantly compress the nerve elements and produce pain, weakness, sensory changes, clumsiness and other manifestation of nervous system dysfunction.

Constriction of the canal within the lumbar spine is termed lumbar stenosis. This condition is common in the elderly and causes a significant proportion of the low back pain, lower extremity pain, lower extremity weakness, limitation of mobility and the high disability rates that afflict this age group. With aging and spinal degeneration, displacement of the vertebral bones in the horizontal may occur and the condition is termed Sponylolisthesis. Spondylolisthesis exacerbates the extent of nerve compression within the spinal canal since misalignment of the vertebral bones will further reduce the size of the spinal canal.

Relief for the compressed nerves can be achieved by the surgical removal of the bone and ligamentous structures that constrict the spinal canal. However, decompression of the spinal canal can further weaken the facet joints and increase the possibility of additional aberrant vertebral movement in the horizontal plane. Thus, decompression can worsen the extent of spondylolisthesis or produce spondylolisthesis in an otherwise normally aligned functional spinal unit. After decompression, surgeons will commonly fuse and immobilize the adjacent spinal bones in order to prevent the development of post-operative vertebral misalignment and spondylolisthesis.

### SUMMARY

Spinal fusion procedures may be substantial operations that carry significant risk and require prolonged post-operative recuperation. Further, vertebral fusion will often place additional load on the adjacent spinal segments and hasten degeneration of those levels. Thus, a long-felt need exists for a less invasive way to address facet joint degeneration while preserving vertebral motion at the degenerated functional spinal unit (FSU). The present invention is a response to this need.

It is a goal of the present disclosure to position an orthopedic implant within the facet joint and attach the implant to the posterior surface of the SAP (superior articular process) of the inferior vertebral bone or onto the anterior surface of the IAP (inferior articular process) of the superior vertebral bone. It is a goal of the disclosure to have the implant increase the distance between the IAP (superior vertebra) and SAP (inferior vertebra) surfaces that make up a facet joint and reduce the joint laxity and at least partially correct the spondylolisthesis produced by the degenerative process.

It is a further goal of the disclosure to provide at least one method that permits minimally invasive placement of the implant. In an embodiment, the device is implanted using a percutaneous technique. In an embodiment, the device forms an osseous or bony bond with the first vertebra. Preferably, but not necessarily, the device contains a cavity into which bone graft material would be placed in order to form a bone fusion mass within the cavity, wherein the mass is also fused with a vertebral bone of the FSU.

In one aspect, there is disclosed an orthopedic implant adapted for implantation within a facet joint of a spinal column, comprising: a smooth abutment surface that is adapted to form a low friction articulation with adjacent bone; a threaded outer surface; an internal cavity adapted to contain bone formation material adapted to form a fusion mass with the bone to which the device is anchored.

In another aspect, there is disclosed a method for the percutaneous repair of a natural function of a facet joint, comprising: localizing a diseased target facet joint using radiographic imaging; positioning a distraction platform using radiographic imaging to engage the spinous processes of a superior and inferior vertebral bone; applying a distractive force to the spinous processes in order to distract the superior and inferior vertebral bone; using vertebral distraction to open the targeted facet joint; opening the facet joint capsule; and inserting an implant into the targeted facet joint, wherein the implant contains a smooth abutment surface that is adapted to form a low friction articulation with adjacent bone, a threaded outer surface, and an internal cavity adapted to contain bone formation material.

Other features and advantages should be apparent from the following description of various embodiments, which illustrate, by way of example, the principles of the disclosed devices and methods.

### BRIEF DESCRIPTION OF THE DRAWINGS

FIG. 1 shows a diagrammatic representation of a spinal vertebral bone in multiple views

FIGS. 2A and 2B illustrate a functional spinal unit (FSU).

FIG. 3A illustrates three vertebral bones with relatively normal alignment.

FIG. 3B shows the anterior displacement of the middle bone relative to the inferior-most bone

FIG. 4A shows a side view of a portion of the spine.

FIG. 4B shows a cross-sectional view of a portion of the spine.

FIG. 5 illustrates perspective and orthogonal views of a first device embodiment.

FIG. 6 shows sectional views of the device.

FIGS. 7 to 11 show a method for percutaneous implantation of the device.

FIG. 12 shows perspective views of an additional embodiment.

FIG. 13 shows orthogonal views of the embodiment of FIG. 12.

FIG. 14 shows sectional views of the embodiment of FIG. 12.

FIGS. 15A and 15B show a threaded fastener with a central cavity.

FIGS. 16A and 16B shows a locking cap.

FIG. 17A illustrates how the device, fastener and cap 335 are arranged at implantation.

FIG. 17B shows an assembled construct.

FIG. 18 shows the implanted device prior to closure of the distraction platform.

FIG. 19 shows the implanted device after closure of the distraction platform.

FIG. 20A shows the cap attached to the fastener 305.

FIG. 20B shows an additional method of use.

FIGS. 21A and 21B shows an implanted fastener.

### DETAILED DESCRIPTION

In order to promote an understanding of the principals of the invention, reference is made to the drawings and the embodiments illustrated therein. Nevertheless, it will be understood that the drawings are illustrative and no limitation of the scope of the invention is thereby intended. Any such alterations and further modifications in the illustrated embodiments, and any such further applications of the principles of the invention as illustrated herein are contemplated as would normally occur to one of ordinary skill in the art.

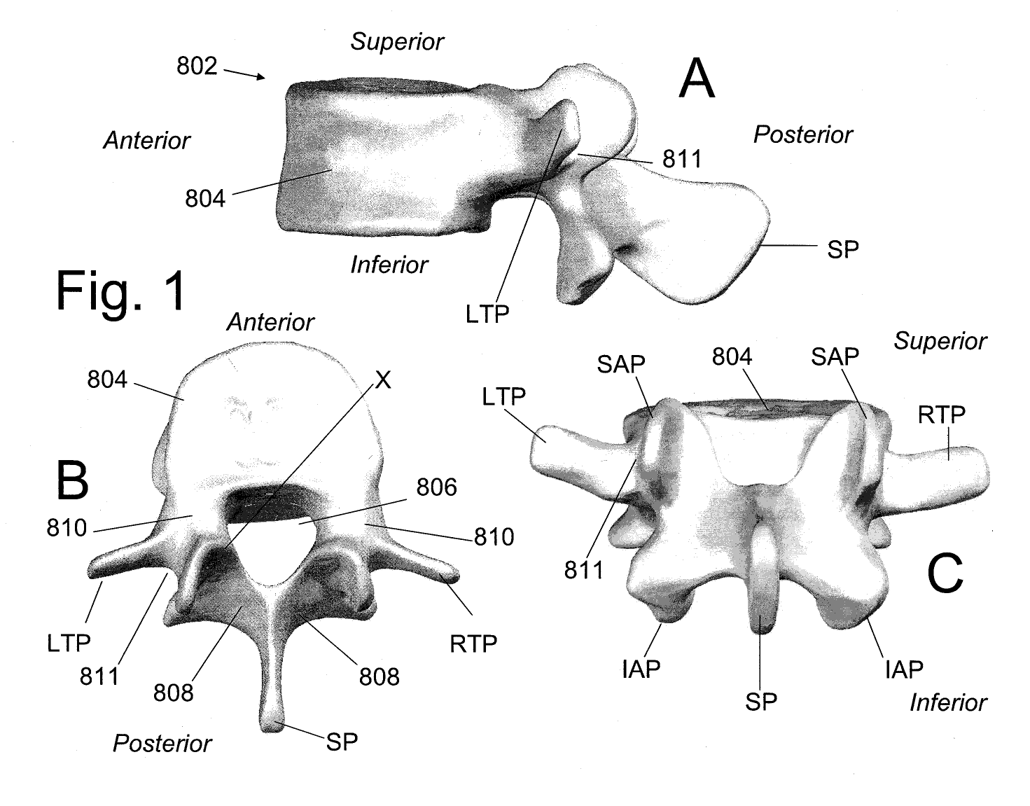

FIG. 1 shows a diagrammatic representation of a spinal vertebral bone 802 in multiple views. For clarity of illustration, the vertebral bone of FIG. 1 and those of other illustrations presented in this application are represented schematically and those skilled in the art will appreciate that actual vertebral bodies may include anatomical details that are not shown in these figures. Further, it is understood that the vertebral bones at a given level of the spinal column of a human or animal subject will contain anatomical features that may not be present at other levels of the same spinal column. The illustrated vertebral bones are intended to generically represent vertebral bones at any spinal level without limitation. Thus, the disclosed devices and methods may be applied at any applicable spinal level.

Vertebral bone 802 contains an anteriorly-placed vertebral body 804, a centrally placed spinal canal and 806 and posteriorly-placed lamina 808. The pedicle (810) segments of vertebral bone 802 form the lateral aspect of the spinal canal and connect the laminas 808 to the vertebral body 804. The spinal canal contains neural structures such as the spinal cord and/or nerves. A midline protrusion termed the spinous process (SP) extends posteriorly from the medial aspect of laminas 808. A protrusion extends laterally from each side of the posterior aspect of the vertebral bone and is termed the transverse process (TP). A right transverse process (RTP) extends to the right and a left transverse process (LTP) extends to the left. A superior protrusion extends superiorly above the lamina on each side of the vertebral midline and is termed the superior articulating process (SAP). An inferior protrusion extends inferiorly below the lamina on each side of the vertebral midline and is termed the inferior articulating process (IAP). Note that the posterior aspect of the pedicle can be accessed at an indentation 811 in the vertebral bone between the lateral aspect of the SAP and the medial aspect of the transverse process (TP). In surgery, it is common practice to anchor a bone fastener into the pedicle portion of a vertebral bone by inserting the fastener through indentation 811 and into the underlying pedicle.

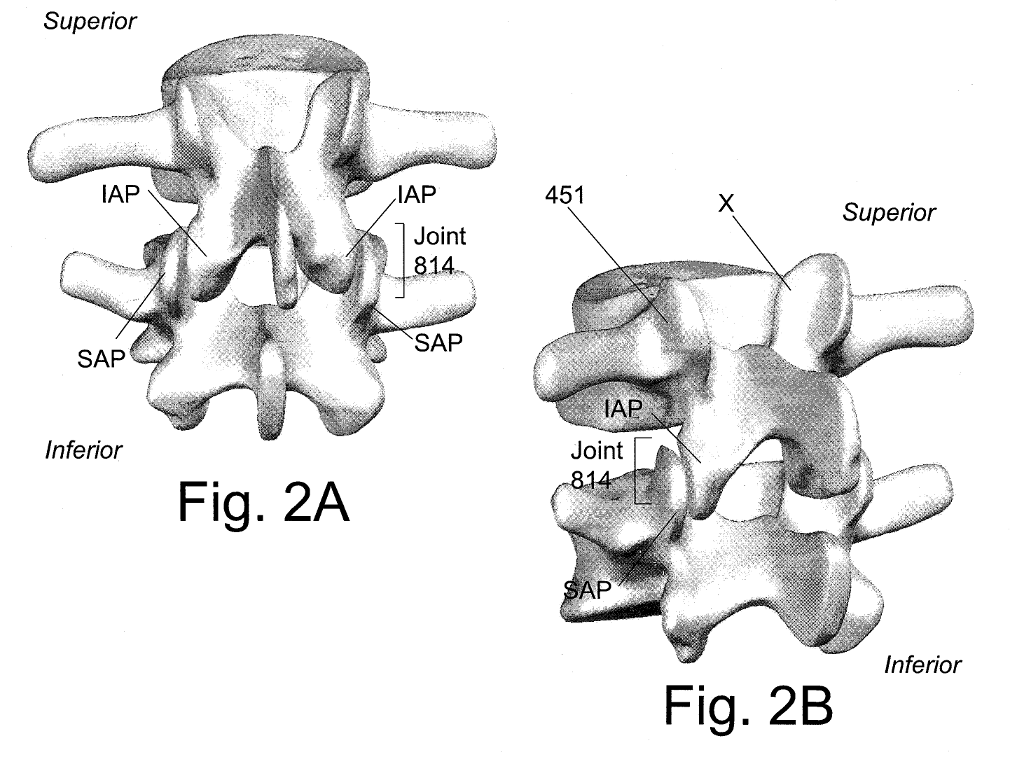

FIGS. 2A and 2B illustrate a functional spinal unit (FSU), which includes two adjacent vertebrae and the intervertebral disc between them. The intervertebral disc resides between the inferior surface of the upper vertebral body and the superior surface of the lower vertebral body. (Note that a space is shown in FIG. 2 where intervertebral disc would reside.) FIG. 2A shows the posterior surface of the FSU vertebrae and the articulations between them while FIG. 2B shows an oblique view. Note that an FSU contains a three joint complex between the two vertebral bones, with the intervertebral disc comprising the anterior joint. The posterior joints include a facet joint 814 on each side of the midline, wherein the facet joint contains the articulation between the IAP of the superior vertebral bone and the SAP of the inferior vertebral bone.

The preceding illustrations and definitions of anatomical structures are known to those of ordinary skill in the art. They are illustrated in more detail in the text Atlas of Human Anatomy, by Frank Netter, third edition, Icon Learning Systems, Teterboro, N.J. and in the text Gray's Anatomy: The Anatomical Basis of Clinical Practice, by Susan Standring, 39^{th} edition, Elsevier, Churchill, Livingstone, New York, N.Y. Each text is hereby incorporated by reference in its entirety.

In a functional spinal unit, a substantial portion (up to 80%) of the vertical load is borne by the intervertebral disc and the anterior column. (The term “vertical load” refers to the load transmitted in the vertical plane through the erect human spine. The “anterior column” is used here to designate that portion of the vertebral body and/or FSU that is situated anterior to the posterior longitudinal ligament and includes the posterior longitudinal ligament. Thus, its use in this application encompasses both the anterior and middle column of Denis. See The three column spine and its significance in the classification of acute thoracolumbar spinal injuries. By Denis, F. Spine 1983 November-December; 8(8):817-31. The article is incorporated by reference in its entirety.) Conversely, a substantial portion of load transmitted through the functional spine unit in the horizontal plane is borne by the facet joint and the posterior column. (The “posterior column” is used here to designate that portion of the vertebral body and/or FSU that is situated posterior to the posterior longitudinal ligament.) Generally, the forces acting in the horizontal plane are aligned to cause an anterior displacement of the superior vertebral body relative to the inferior vertebral body of a functional spinal unit. These forces are counteracted by the abutment, the facet joints, the limit anterior movement of the superior vertebral relative to the inferior vertebral bone. As previously noted, the facet joints are formed by the abutment of the IAP of the superior vertebral bone and the SAP of the inferior bone.

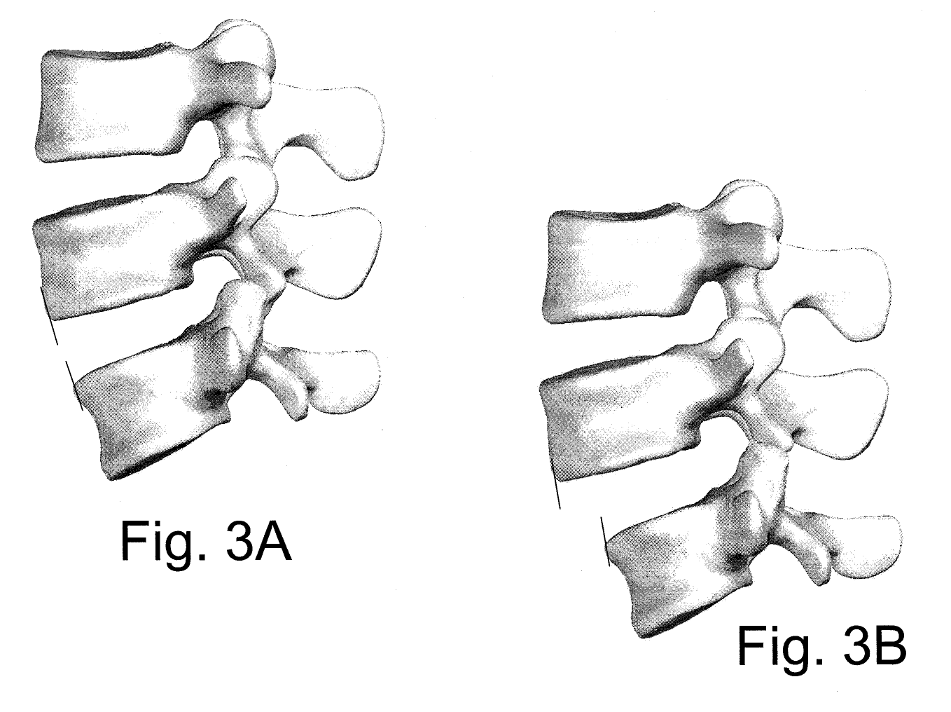

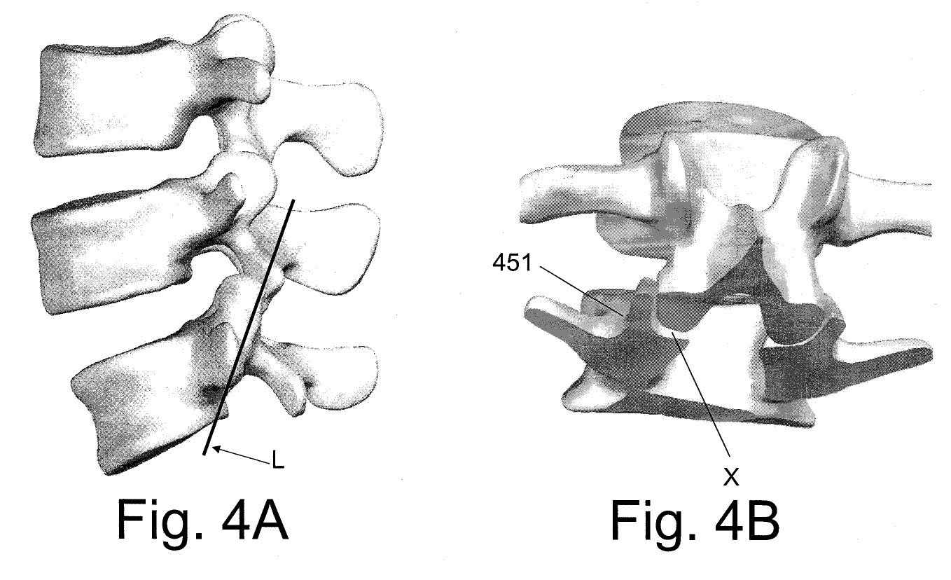

In a healthy spine functioning within physiological parameters, the two facet joints of an FSU collectively function to prevent aberrant relative movement of the vertebral bones in the horizontal plane. With aging and spinal degeneration, displacement of the vertebral bones in the horizontal may occur and the condition is termed Sponylolisthesis. FIG. 3A illustrates three vertebral bones with relatively normal alignment, whereas FIG. 3B shows the anterior displacement of the middle bone relative to the inferior-most bone. In the illustration, the vertebral column of FIG. 3B is said to have an anterior spondylolisthesis of the middle vertebral bone relative to the inferior-most vertebral bone, since the middle bone is anteriorly displaced relative to the inferior bone.

A spondylolisthesis can be anterior, as shown in FIG. 3B, or posterior wherein a superior vertebral bone of a functional spinal unit is posteriorly displaced in the horizontal plane relative to the inferior vertebral bone. Anterior Sponylolisthesis is more common and more clinically relevant than posterior Sponylolisthesis. (Sponylolisthesis can be further classified based on the extent of vertebral displacement. See Principles and practice of spine surgery by Vaccaro, Bets, Zeidman; Mosby press, Philadelphia, Pa.; 2003. The text is incorporated by reference in its entirety.)

With degeneration of the spine, constriction of the spinal canal (spinal stenosis) and impingement of the contained nerve elements frequently occurs and is termed spinal stenosis. Spondylolisthesis exacerbates the extent of nerve compression within the spinal canal since misalignment of bone within the horizontal plane will further reduce the size of the spinal canal. Further, the facet joints in spondylolisthesis patients are often lax such that the IAP and SAP abutment surfaces are not well opposed and aberrant vertebral displacement in the horizontal plane is permitted with flexion and extension of the FSU.

Relief for the compressed nerves can be achieved by the surgical removal of the bone and ligamentous structures that constrict the spinal canal. However, decompression of the spinal canal can further weaken the facet joints and increase the possibility of additional aberrant vertebral movement in the horizontal plane and worsen the extent of spondylolisthesis or produce spondylolisthesis in an otherwise normally aligned FSU. After decompression, surgeons will commonly fuse and immobilize the adjacent spinal bones in order to prevent the development of post-operative vertebral misalignment and spondylolisthesis.

Spinal fusion procedures may be substantial operations that carry significant risk and require prolonged post-operative recuperation. Further, vertebral fusion will often place additional load on the adjacent spinal segments and hasten degeneration of those levels. Thus it is a goal of the present invention to position an orthopedic implant within the facet joint and attach the implant to the posterior surface of the SAP of the inferior vertebral bone or onto the anterior surface of the IAP of the superior vertebral bone. It is a goal of the invention to have the implant increase the distance between the IAP (superior vertebra) and SAP (inferior vertebra) surfaces that make up a facet joint and reduce the joint laxity and at least partially correct the spondylolisthesis produced by the degenerative process.

It is a further goal of the disclosure to provide at least one method that permits minimally invasive placement of the implant. In an embodiment, the device is implanted using a percutaneous technique. The device may form an osseous or bony bond with the first vertebra. The device may contain a cavity into which bone graft material (i.e., a material adapted to form bone such as bone fragments, synthetic bone graft substitutes, growth factors that are capable of promoting and forming bone, and the like) would be placed in order to form a bone fusion mass within the cavity, wherein the mass is also fused with a vertebral bone of the FSU. The device may also contain a surface that can form a direct osseous bond with a vertebral bone of the FSU. (For example, a device surface may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening.).

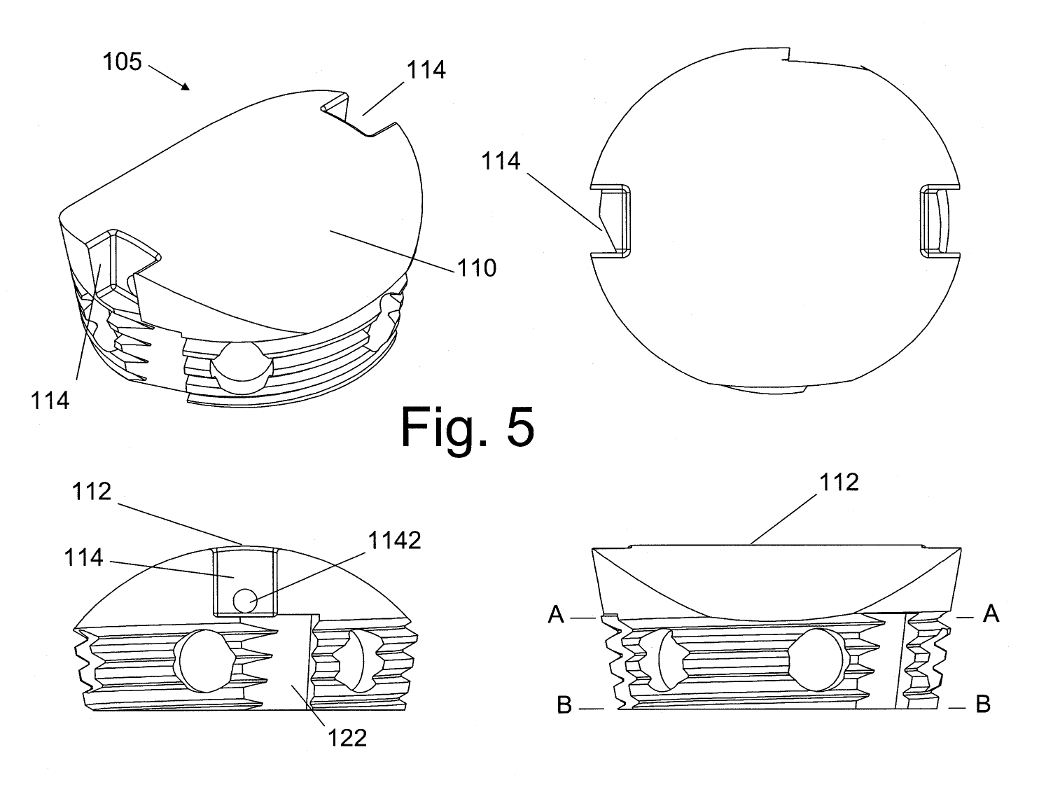

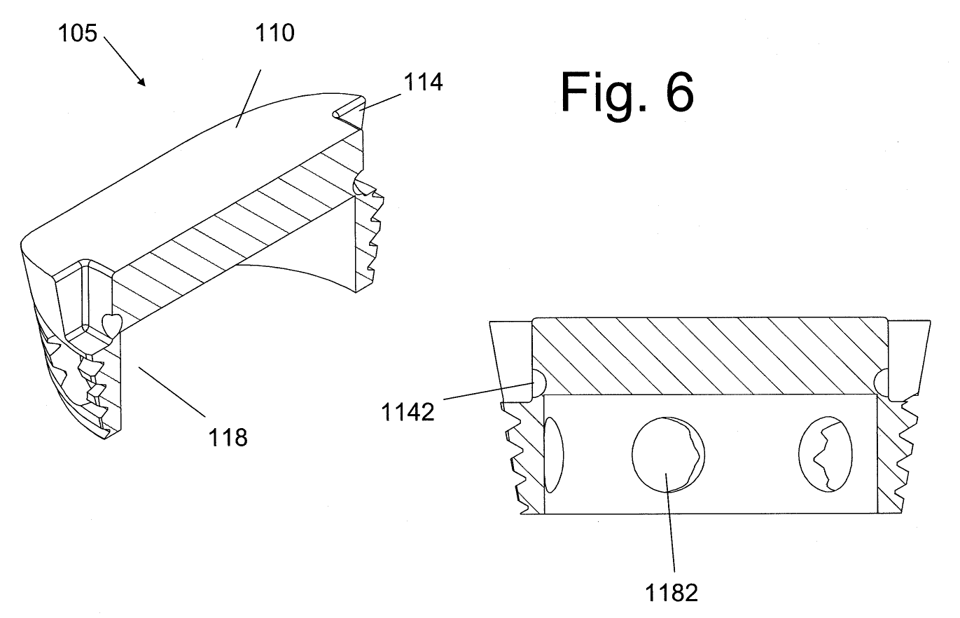

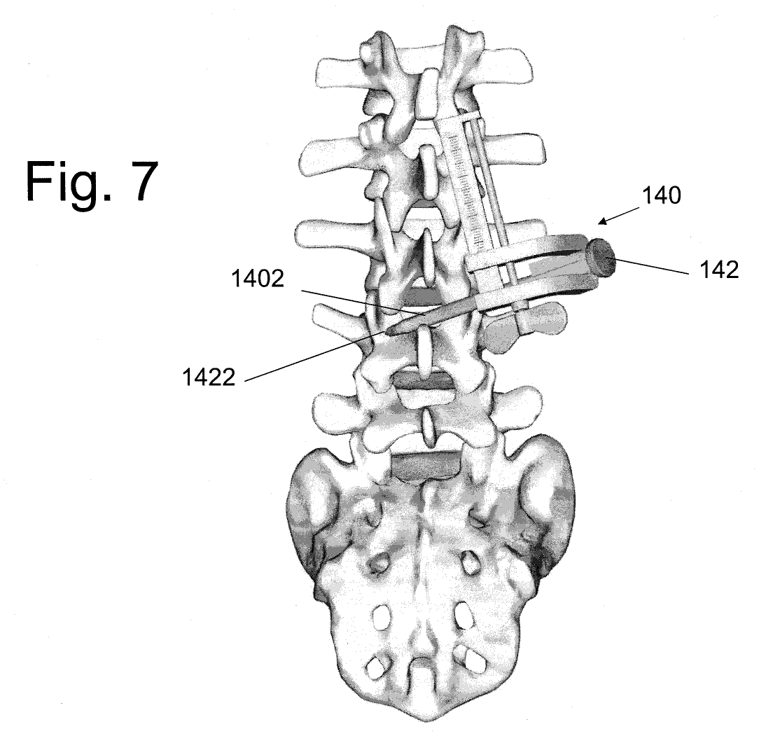

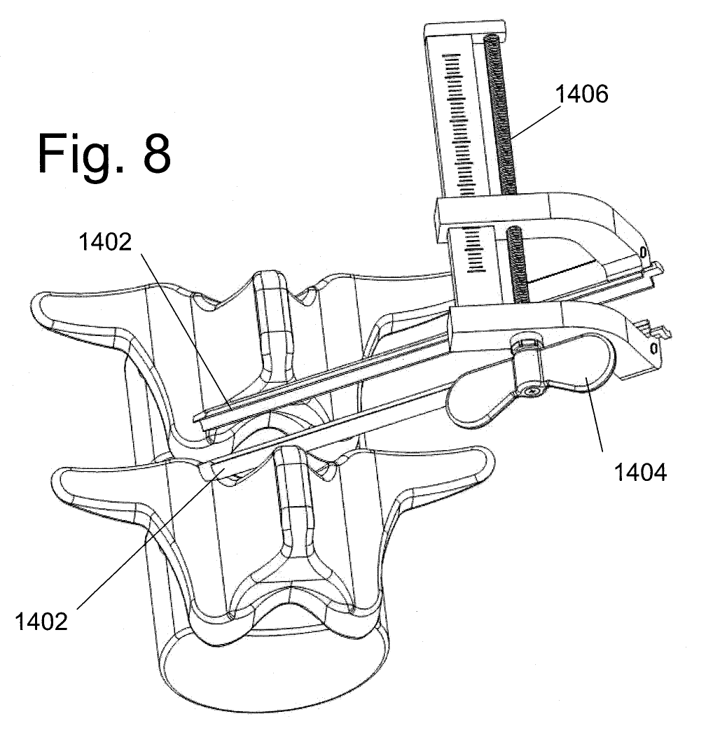

A section view through line L of FIG. 4A is shown in FIG. 4B. Note that the facet joint articulation is comprised of the concave surface of the SAP of the inferior vertebral bone is concave and the convex surface of the IAP of the superior vertebral bone. FIG. 5 illustrates perspective and orthogonal views of a first device embodiment. FIG. 6 shows sectional views.

Device 105 is preferably cylindrical in configuration, wherein the diameter at level of plane A (FIG. 5) is greater that the diameter at the level of plane B. The device segment between planes A and B is preferably threaded. An abutment surface 110 is positioned atop the threaded segment. While shown as a convex surface, the device may be alternatively configured to contain an abutment surface of any relevant geometric shape that would provide an effective abutment surface in the facet joint to be implanted. The convex abutment surface 110 contains ridge 112. Side indentations 114 provide attachment points for the instruments used to deliver and position device 105 at the implantation site (implantation instruments are not shown). Further, an additional indentation 1142 is disposed within indentation 114 and adapted to interact with the implantation instruments. While not illustrated, it is contemplated that the implantation instrument would preferably contain at least two protrusions, wherein each protrusion couples to indentations 114 of device 105. A compressive force applied by the implantation instrument so that the protrusions are forced towards one another and device 105 is forcibly captured there between. In this way, device 105 is coupled to the implantation instrument.

Abutment surface is preferably a polished smooth that provides a low friction articulation with the segment of bone that abuts and articulates with it. Internal cavity 118 is contained within device 105, wherein the cavity is adapted to contain a bone forming material, such as a bone graft or bone graft substitute. The outer walls of cavity 118 may contain bore holes 1182 that permit communication between the bone graft material contained within cavity 118 and the bone into which the device is threadedly attached (that is, the vertebral bone outside of cavity 118). Partial thickness indentations 122 may be present on the threaded portion of the device in order to promote and hasten the advancement of the threads into bone.

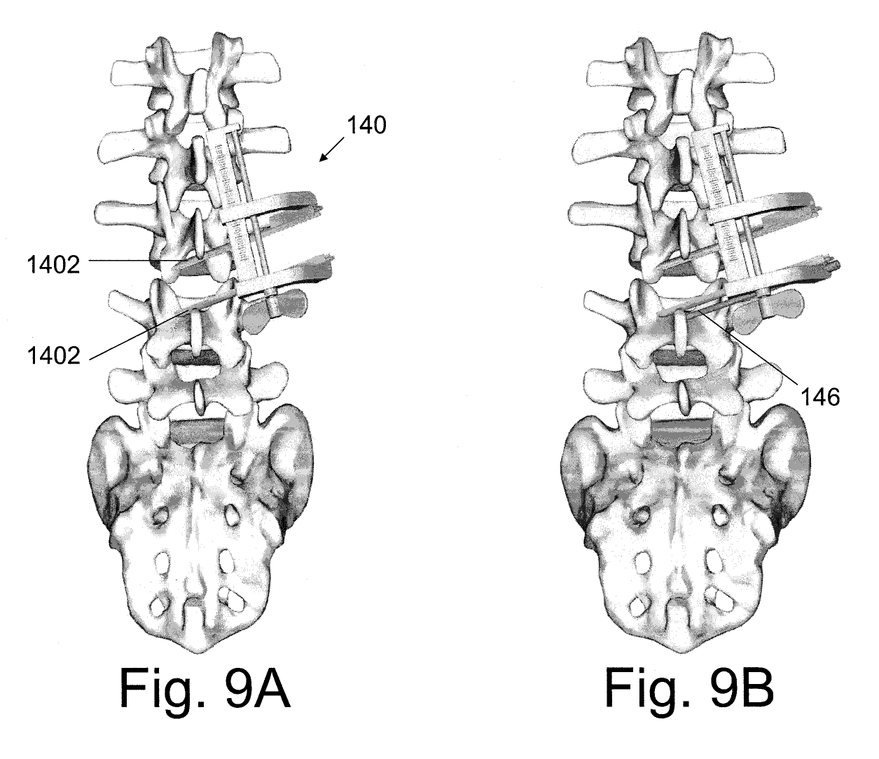

A method for percutaneous implantation is shown in FIGS. 7 to 11. The level of implantation is identified on X-ray. Preferably, fluoroscopy is used to identify the operative level. Fluoroscopy is used through out the procedure and, preferably, in at least two orthogonal planes. The right and left facet joints at the level of implantation are identified and the facet joint to be implanted first is targeted. A distraction platform is percutaneously inserted from the contra-lateral side. (That is, the distraction platform is inserted through the skin (skin not shown for diagrammatic simplicity) at a site that is across the vertebral midline from the targeted facet joint.) The distal end of the cylindrical central port 1402 with central trocar 142 is aimed towards the facet joint that will be implanted. The distal end of port 1402 and tip 1422 of trocar 142 are forcibly advanced through the interspinous tissues and towards the medial aspect of the targeted facet joint. The trocar is removed and the distraction platform is actuated in order to distract the superior and inferior vertebral bones of the FSU to be implanted.

The distraction platform is shown in FIG. 8. The platform has a cylindrical central port 1402 that separates into two semi-cylindrical sections with actuation of thumb screw 1404—which is used to drive threaded lead screw 1406. A central trocar 142 with sharpened tip 1422 is adapted to reside within the internal channel of closed central port 1402.

The trocar is placed within port 1402 and used to during device advancement through the body's tissues. After the platform is delivered to the intended target position, the trocar is removed and the thumb screw 1404 is actuated. The opened central hollow cavity within cylindrical central port 1402 forms an open conduit through which the implant may be delivered. In FIG. 8, the platform is shown in the distracted position. The vertebral bones in FIG. 8 are represented schematically and those skilled in the art will appreciate that actual vertebral bodies may include anatomical details that are not shown in the drawing. (A similar distraction is disclosed in US Patent Application Publication number 2008/0027438. The referenced patent application is hereby incorporated by reference in its entirety. An alternative method of spinous process distraction may be used. The method uses spinous process screws and is disclosed in US Patent Application Publication Number 2006/0149278. The disclosure of the cited patent application is hereby incorporated by reference in its entirety.)

An elongated screw 146 may be used to anchor the distraction platform to at lease one spinous process. FIG. 9 show the distraction platform prior to screw placement (FIG. 9A) and after screw placement (FIG. 9B). While FIG. 9 show that the vertebral bones had been distracted prior to placement of screw 146, screw placement may be alternatively performed prior to bone distraction.

Vertebral distraction is performed under fluoroscopic visualization. The joint capsule of the targeted facet is opened and the internal aspect of the joint is accessed. While distraction alone may suffice to expose the medial surface of the SAP of the inferior bone, it is contemplated that an inferior portion of the IAP of the superior bone may be also removed to enhance the exposure of the medical aspect of the SAP. The appropriate size of the device and the optimal implantation location is chosen based on the fluoroscopic image. With distraction, port 1402 opens into two semi-cylindrical segments and forms an internal portal through which an implant may be delivered to the targeted facet joint.

A pilot hole that is smaller than the implant is drilled at the implantation location. Bone fragments may be removed from the drill bite and used as part of the material packed into cavity 118 of device 105. Cavity 118 is packed with bone forming material and the device is threaded and advanced into the predrilled hole. Note that pre-drilling the hole may not be necessary, since indentations 112 may permit the device to function as a self drilling implant. The implant is advanced into the SAP of the inferior vertebral of the targeted facet joint and aligned so that ridge 112 of surface 110 rests in a substantially horizontal plane relative to the long axis of the spine (FIG. 10A). In this way, the apical portion of the convex surface of the IAP of the superior vertebral bone forms a substantially cross-shaped or cruciate abutment with ridge 112 of surface 110 of the implant. The configuration is schematically shown in cross-section in FIG. 11B.

The distraction platform is removed to leave the spine as shown in FIG. 10B. While the procedure may be performed on one side of a spinal level, it is preferably performed bilaterally at the diseased levels. After bilateral implantation, the implanted spine appears as schematically shown in FIG. 11A. With time, the contents of cavity 118 will fuse with the surrounding bone and solidly anchor the device to the vertebral bone. The implanted devices will restore an abutment surface between the upper and lower fact surfaces of a functional spinal unit. The devices will reduce laxity of a degenerated facet joint and may at least partially correct the spondylolisthesis produced by the degenerative process. While the implant is shown attached to the SAP of the inferior vertebral bone, it may be alternatively attached to the IAP of the superior vertebral bone alone or to both SAP and IAP of a facet joint.

In an alternative device embodiment, device 105 may made without cavity 118. In an alternative method of vertebral distraction (not illustrated), a balloon is positioned between the spinous processes of the superior and inferior vertebral bone. The inflated balloon is used to distract the vertebral bones during implantation. After device implantation, the balloon is removed.

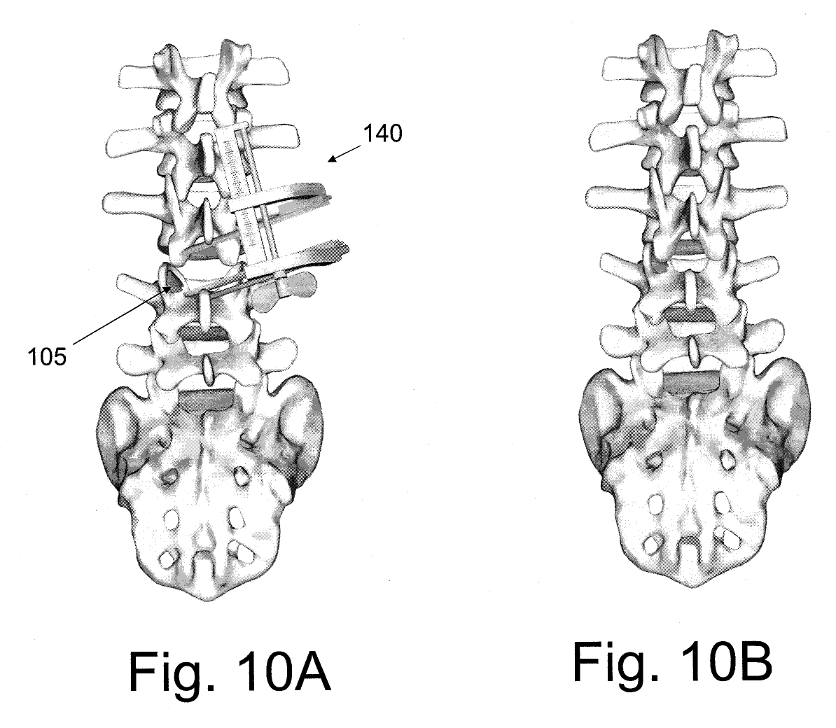

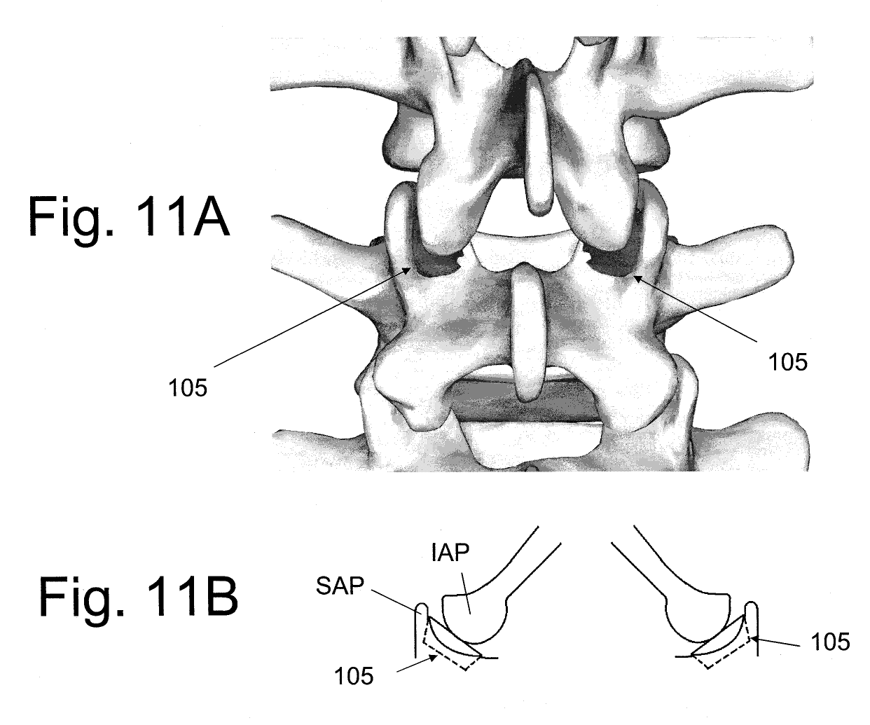

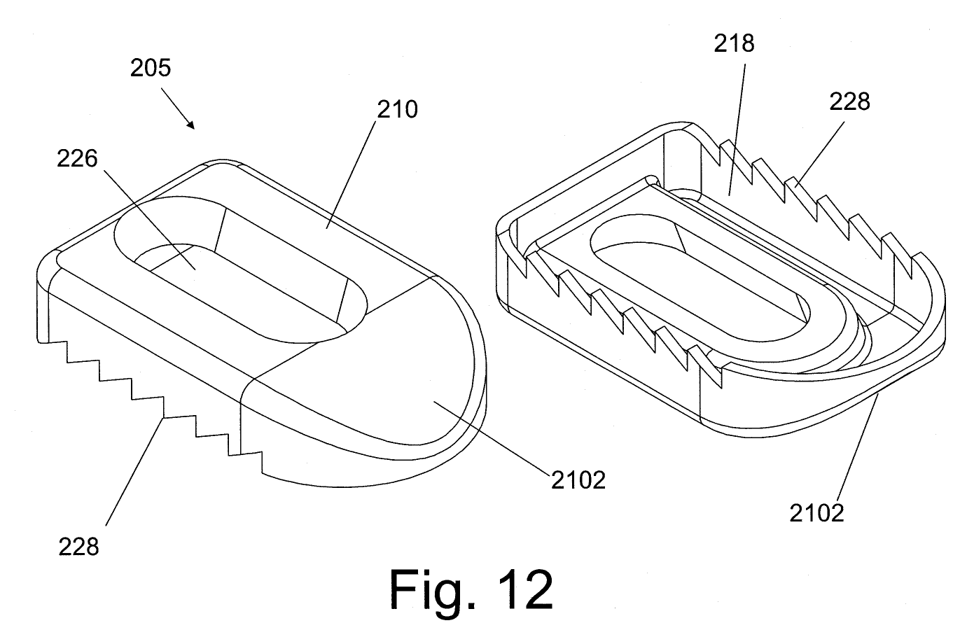

Perspective views of an additional embodiment are shown in FIG. 12. Orthogonal views are shown in FIG. 13 and section views are shown in FIG. 14. Device 205 has as abutment surface 210 with curvilinear leading edge 2102. Internal cavity 218 is adapted to contain a bone forming material. A slot 226 is adapted to accept a bone fastener. Preferably, the walls of slot 226 are angled so that the fastener may be positioned at variable angles/trajectories relative to the slot. Spiked protrusions 228 provide additional fixation to the underlying bone.

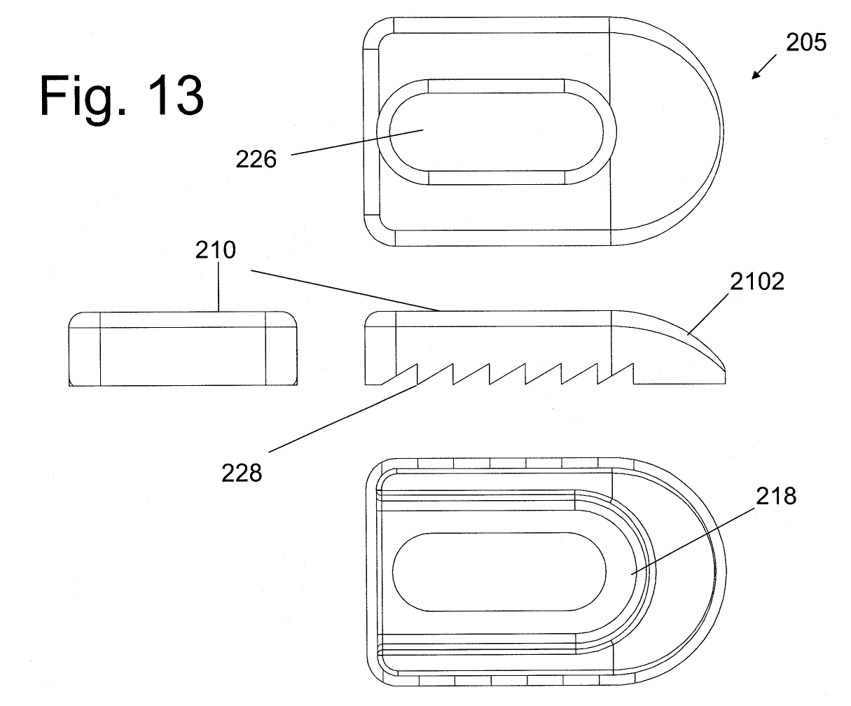

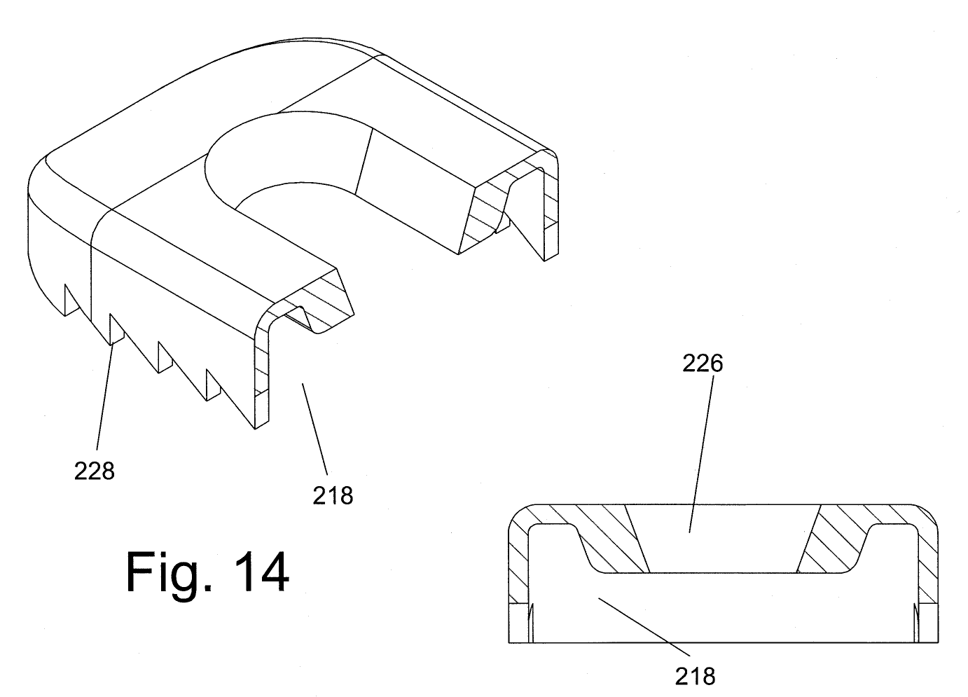

FIG. 15 show a threaded fastener 305 with a central cavity 318. Cavity 318 is adapted to contain bone forming material. The bone material can communicate with the external vertebral bone through bore holes 320 and form a fusion mass with the vertebral bone. A locking cap 335 is shown in FIG. 16A. Cap 335 is has external threads 338 that are adapted to interact with threads 325 of fastener 305. Note that threads 338 are sized to fit through slot 226 whereas fastener 305 is preferably sized to be larger than slot 226.

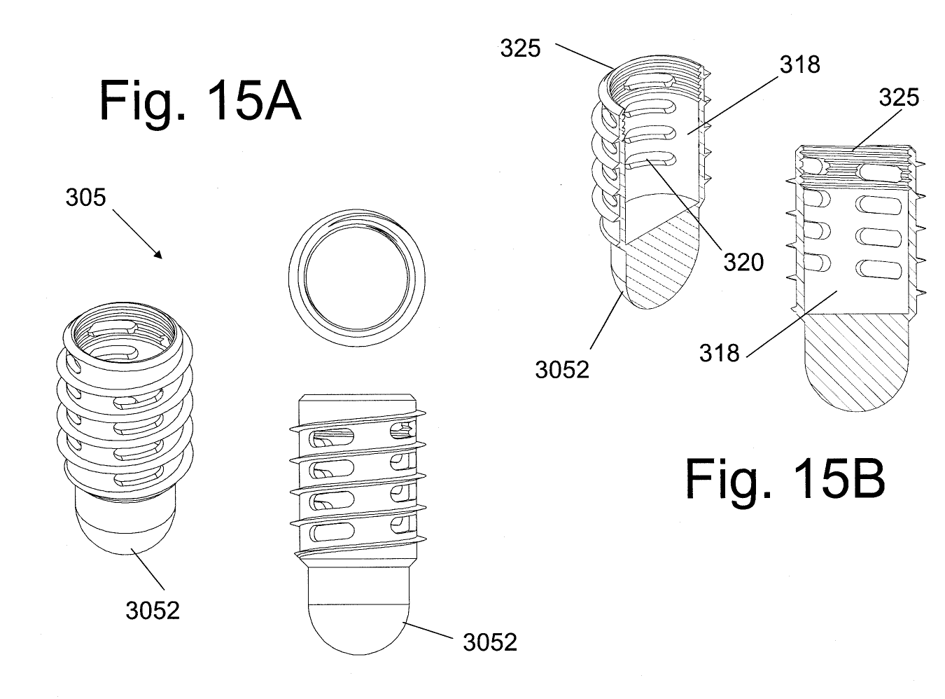

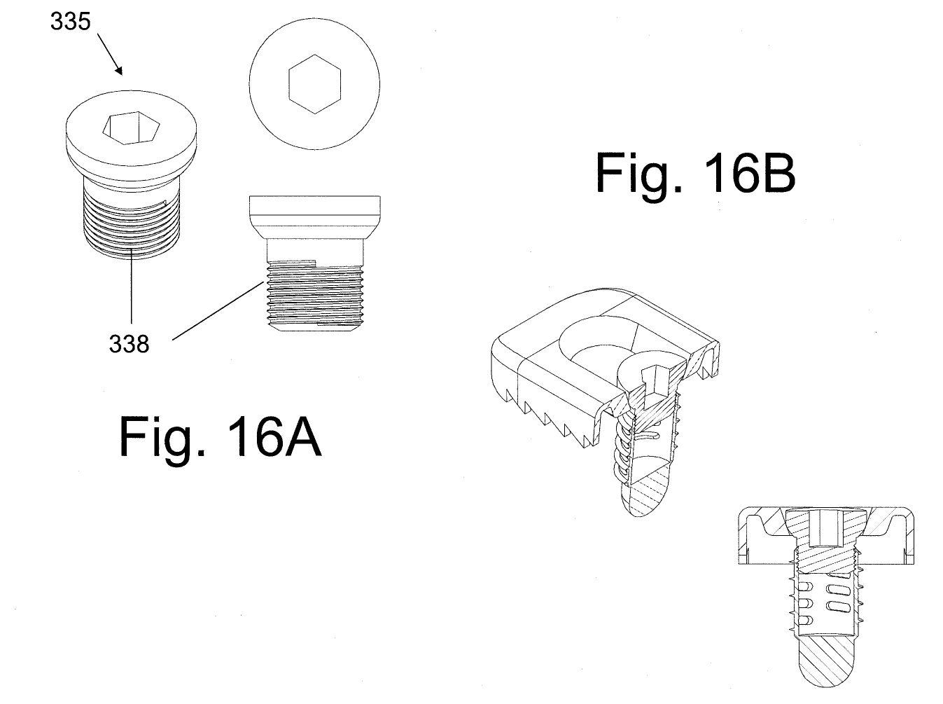

FIG. 17A illustrates how device 205, fastener 305 and cap 335 are arranged at implantation whereas FIG. 17B shows the assembled construct. A section view of the assembly is shown in FIG. 16B. At implantation, a distraction platform is used to percutaneously access the targeted facet joint as previously described. While distraction alone may suffice to expose the medial surface of the SAP of the inferior bone, it is contemplated that an inferior portion of the IAP of the superior bone may be also removed to enhance the exposure of the medical aspect of the SAP.

The cartilaginous surface of the SAP of the inferior bone is removed and the underlying bone is decorticated in preparation for fusion with the bone forming material within cavity 218 of device 205. (Methods of cartilage removal and bone decortications are well known in the art and include use of scrapping tools such as curettes, wire brushes and the like.) Cavity 318 of fastener 305 is packed with bone forming material and the fastener is advanced into the inferior vertebra at or about the region of the SAP (and, possibly, into the underlying pedicle). Device 205 is positioned onto the medial surface of the SAP of the inferior vertebral bone and advanced superiorly until it rests in the desired position relative to the IAP of the superior vertebra bone. Cap 335 is then used to rigidly affix device 205 onto the SAP and anchor it to the fastener 305. Spiked protrusions 228 provide additional fixation to the underlying bone. With time, the contents of cavities 218 and 318 will fuse with the surrounding bone and solidly anchor the device to the inferior vertebra.

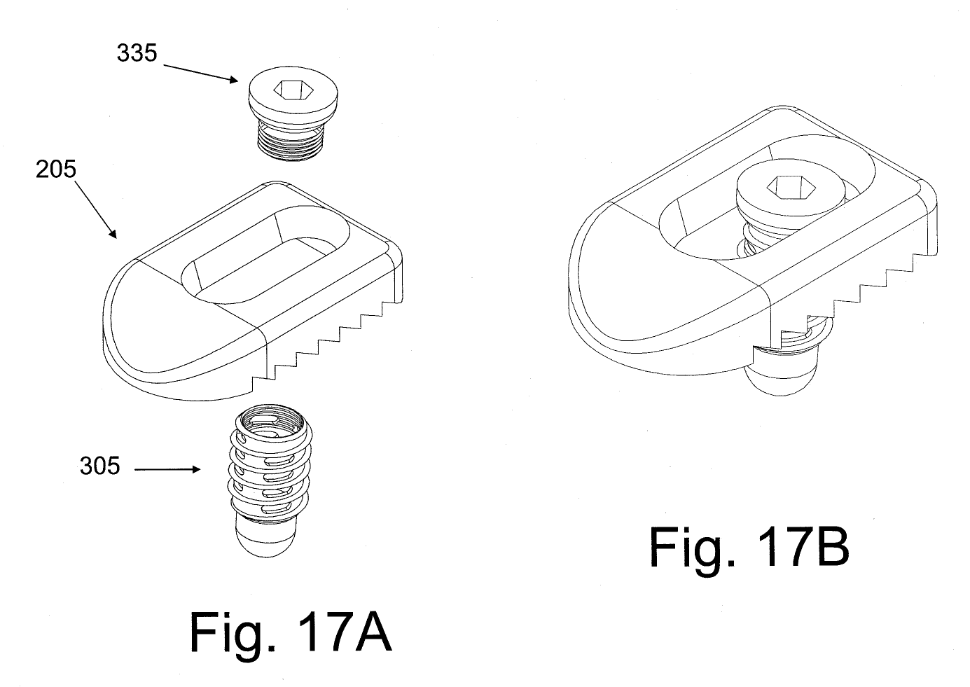

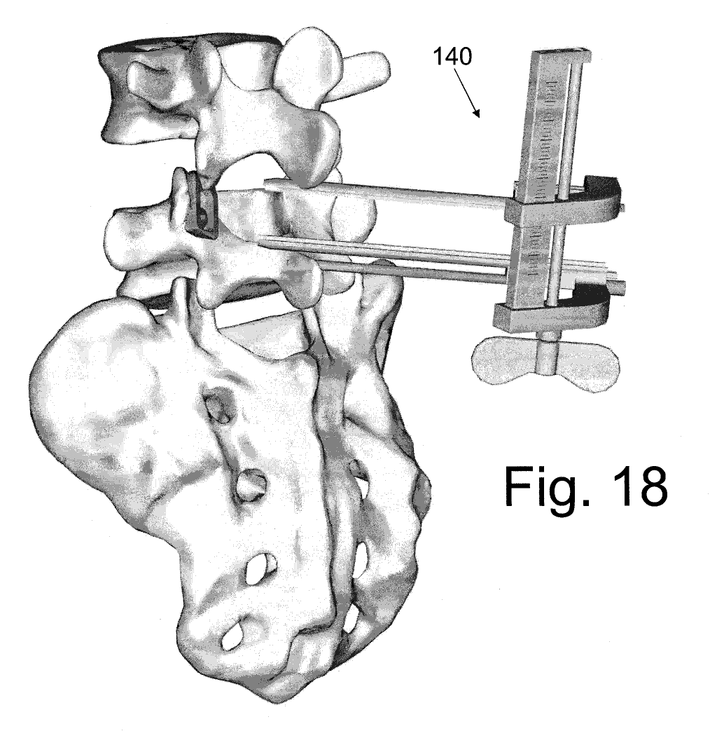

During implantation, slot 226 permits movement of device 205 (and the abutment surface) relative to fastener 305. The surgeon can move device 205 until the abutment surface is optimally placed relative to the IAP of the superior vertebral bone. After the engagement of locking cap 335, the device is immobilized relative to the inferior vertebral bone. Further, the orientation of spiked protrusions 228 will resist the inferior migration of device 205. The implanted device is shown in FIG. 18 prior to closure of the distraction platform and in FIG. 19 after closure of the distraction platform.

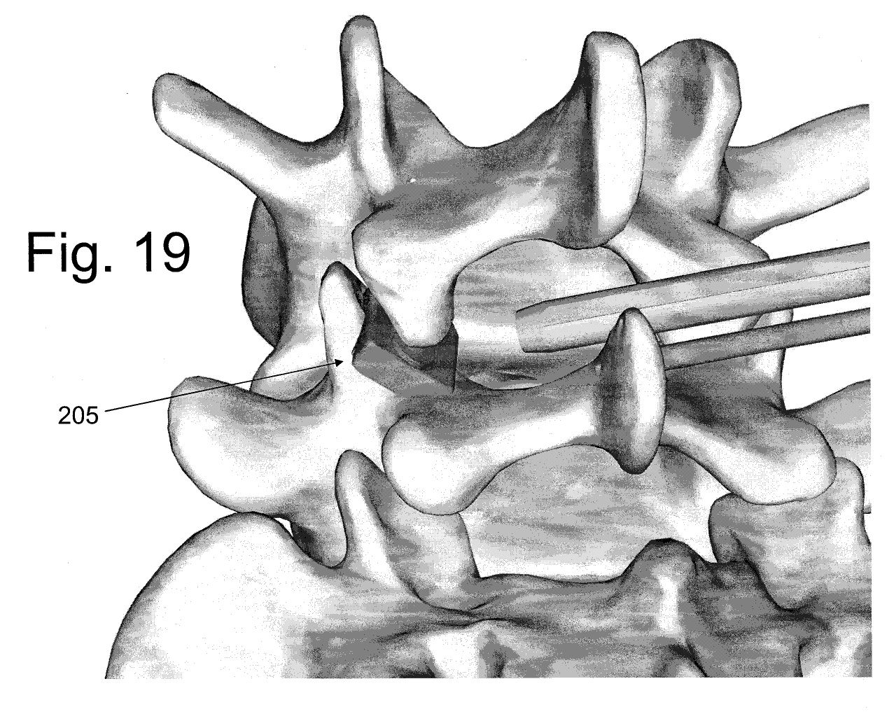

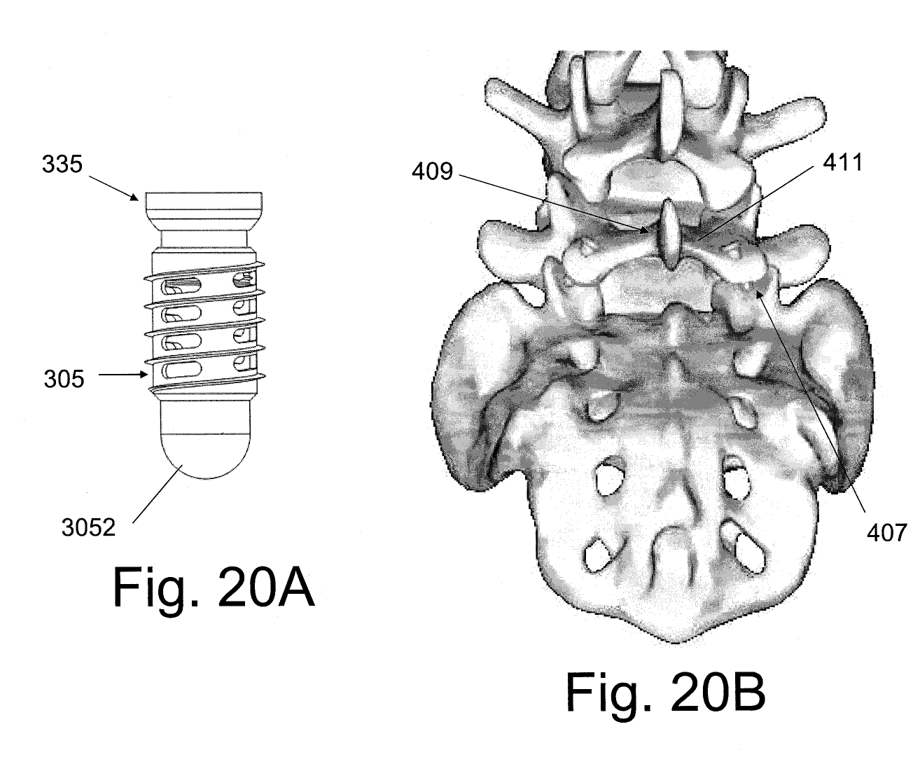

An additional method of use is shown in FIG. 20B. Central cavity 318 of fastener 305 is filled with bone forming material and cap 335 is attached to fastener 305—as shown in FIG. 20A. (Note that while the method is described using a fastener that contains a central cavity 318, a solid fastener may be alternatively used—as would be obvious to one of ordinary skill in the art.) A bore hole is placed into the posterior aspect of the lamina of the superior vertebral bone of the FSU to be implanted. Fastener 305 with cap 335 is then advanced through the pre-formed bore so that end 3052 of the fastener emerges from the IAP of the superior vertebral bone and enters the facet joint. The fastener is advanced until surface 3052 abuts—but does not penetrate—the posterior surface of the SAP of the inferior vertebral bone. Using this method, an abutment is formed between surface 3052 of fastener 305 and the SAP of the inferior vertebra. With time, the contents of cavity 318 will fuse with the surrounding bone of the ipsi-lateral lamina and solidly anchor the fastener to the superior vertebral bone. The method can be used to increase the distance between the IAP (superior vertebra) and SAP (inferior vertebra) surfaces that make up the diseased facet joint, reduce the joint laxity and may at least partially correct a spondylolisthesis that was produced by the degenerative process. Further, the totality of fastener placement can be performed using percutaneous techniques—since percutaneous placement of fasteners into the lamina of a vertebral bone is currently known in the art. However, no prior art, taken either singly or in combination, describes the use of fastener to form an abutment surface within a facet joint as disclosed herein.

The prior method of use describes a fastener 305 being introduced into the superior vertebral lamina at a point that was ipsi-lateral (i.e., on the same side of the vertebral midline) to the targeted facet joint. However, a longer fastener may be alternatively introduced into the superior vertebral bone at a point that is contra-lateral to the targeted facet joint. For example, if the facet 407 of FIG. 20B is targeted for implantation, the fastener may be placed into the superior vertebral bone at or about point 409—which is positioned at the intersection of the medial contra-lateral lamina and spinous process. The fastener 305 is advance within the substance of the bone of the ipsi-lateral lamina 411 until end surface 3052 enters joint 407 and abuts the SAP of the inferior vertebral bone. With time, the contents of cavity 318 will fuse with the surrounding bone of the ipsi-lateral lamina and solidly anchor the fastener to the superior vertebral bone. (See Translaminar Facet Screw Fixation, by Sasso R C and Best N M in World Spine Journal 2006; 1(1): 34-39. The article is incorporated by reference in its entirety.)

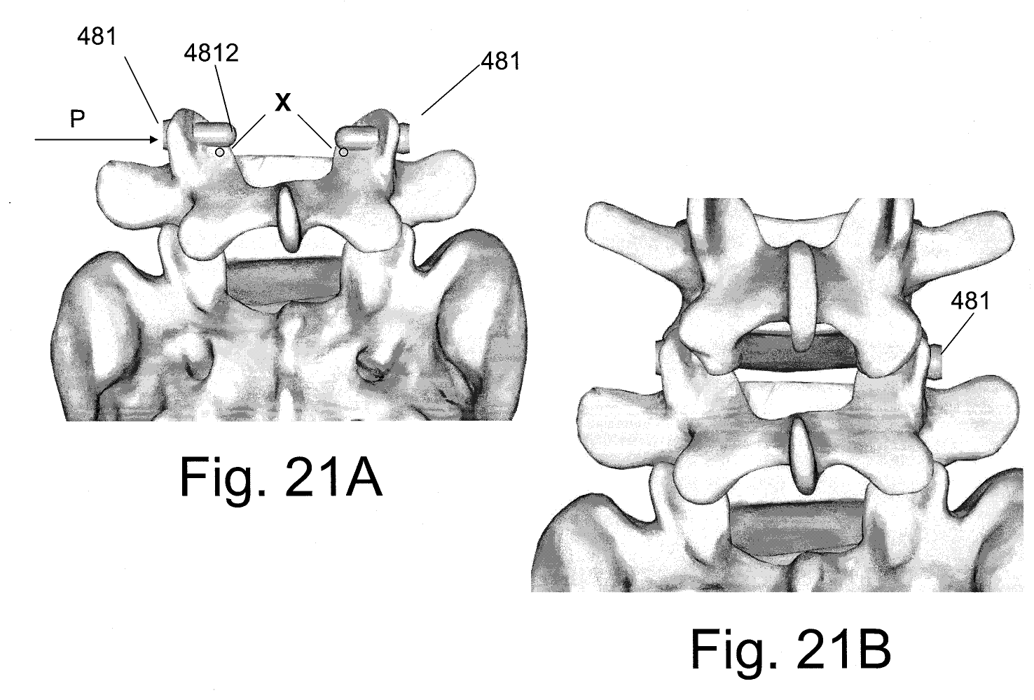

In an alternative method of use, a fastener 481 may be positioned into the facet joint through a bore hole placed into the lateral wall of the SAP of inferior vertebral bone. The fastener entry point is at or about point 451 of FIG. 2B. The faster enters the lateral wall of the SAP of inferior vertebral bone using a directly lateral (See “P” in FIG. 21A) or obliquely lateral approach. Alternatively, the fastener may be positioned into the lateral wall of the SAP via a curvilinear approach, wherein the fastener is positioned through a curvilinear port onto the lateral aspect of SAP of the inferior vertebra. The curvilinear port is inserted through the patient's skin at a point posterior to the spinous process of the spine and lateral to the targeted facet joint. The curvilinear port then follows a curvilinear path through the soft tissues that border the spine and onto the lateral aspect of the SAP. In this way, the fastener, when positioned at the proximal aspect of the curvilinear port (which is preferably outside the patient's body), has a long axis that is aligned in a substantially anterior to posterior orientation. The fastener, when it emerges from the distal aspect of the port (within the patient's body and at the lateral aspect of the SAP of the inferior vertebra) is aligned in a substantially lateral to medial orientation. (An example of a curvilinear insertion port is disclosed in US Patent Application Publication Number 2006/0149278. The disclosure of the cited patent application is hereby incorporated by reference in its entirety.) Fastener 481 may be of any appropriate design known in the art—including linear an curvilinear fasteners. The fastener may be a solid fastener or it may contain a central cavity that is adapted to contain bone graft material, as shown in the embodiment of FIG. 20A. An implanted fastener is shown in FIGS. 21A and 21B. While the fastener is shown as positioned substantially within the horizontal plane, the fastener may be alternatively angled—such as, for example, in the horizontal or vertical planes. In an embodiment, the distal aspect 4812 of fastener 481 may be angled towards point “X” of the anterior aspect of the SAP so that the distal aspect 481 of the fastener enters the SAP at or about point “X”.

The disclosed devices or any of their components can be made of any biologically adaptable or compatible materials. Materials considered acceptable for biological implantation are well known and include, but are not limited to, stainless steel, titanium, tantalum, combination metallic alloys, various plastics, resins, ceramics, biologically absorbable materials and the like. Any components may be also coated/made with nanotube materials to further impart unique mechanical or biological properties. In addition, any components may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation. Further, any surface may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. Lastly, any disclosed devices or any of its components can also be entirely or partially made of a shape memory material or other deformable/malleable material.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. An orthopedic implant adapted for implantation within a facet joint of a spinal column, comprising:
a smooth abutment surface that is adapted to form a low friction articulation with adjacent bone;
a threaded outer surface;
an internal cavity adapted to contain bone formation material adapted to form a fusion mass with the bone to which the device is anchored.

2. A method for the percutaneous repair of a natural function of a facet joint, comprising:
localizing a diseased target facet joint using radiographic imaging;
positioning a distraction platform using radiographic imaging to engage the spinous processes of a superior and inferior vertebral bone;
applying a distractive force to the spinous processes in order to distract the superior and inferior vertebral bone;
using vertebral distraction to open the targeted facet joint;
opening the facet joint capsule; and
inserting an implant into the targeted facet joint, wherein the implant contains a smooth abutment surface that is adapted to form a low friction articulation with adjacent bone, a threaded outer surface, and an internal cavity adapted to contain bone formation material.
